(19) United States
(12) Patent Application Publication
(10) Pub. No.: US 2012/0041293 A1
(43) Pub. Date: Feb. 16, 2012

(54) METHODS AND DEVICES FOR PROCESSING PULSE SIGNALS, AND IN PARTICULAR NEURAL ACTION POTENTIAL SIGNALS

(75) Inventors: Timothée Levi, Leognan (FR); Jean-François Beche, Montbeliard (FR); Stéphane Bonnet, Lyon (FR); Ricardo Escola, Bahia Blanca (AR)

(73) Assignee: COMMISSARIAT A L'ENERGIE ATOMIQUE ET AUX ENE ALT, Paris (FR)

(21) Appl. No.: 13/141,907

(22) PCT Filed: Dec. 23, 2008
(86) PCT No.: PCT/IB08/03758

§ 371 Date: Nov. 2, 2011

(51) Int. Cl. A61B 5/05; G06F 19/00

(52) U.S. Cl. 600/377; 702/19

(57) ABSTRACT

A method for estimating a level of noise affecting a sampled and digitized pulse signal, such as a neural action potential signal; detecting signal pulses by thresholding, the threshold being determined by estimating a level of noise according to the method; and classifying thus-detected signals. An implantable device can carry out the methods. The method and device are applicable to the field of embedded signal processing for multiple electrode arrays implanted in a neural tissue such as a brain.

The invention relates to methods and devices for pulse signals processing, and in particular for processing neural action potential signals (or “spikes”).

More specifically, the invention relates to methods and devices for determining the noise level (e.g. the standard deviation of noise) of an electrophysiological signal acquired by a multi-electrode array implanted in brain tissues, for detecting and extracting “spikes” from such a signal, and for classifying said “spikes”, e.g. in order to associate each spike to an individual neuron. The methods of the invention can be easily implemented in a simple, compact and low-power digital architecture, suitable to be employed in an implantable system.

The term “spikes” refers to pulses representing neuron action potential signals, generally in a 300 Hz-3 kHz frequency band.

Multi-electrode array (MEA) systems used in neurological applications produce large amount of data because of the simultaneous continuous sampling of a large number of channels, e.g. 64 channels, but possibly many more. In typical applications, the input signals from a MEA are continuously recorded at sampling frequencies in the range of 10 kSps-50 kSps (SpS: Samples per Second). Assuming a 12.8 kSps sampling frequency and a 12-bit digital representation of samples, the data rate generated is 153.6 kbit/s/electrode, i.e. 9.6 Mbit/s for a 64 electrode system.

While in in-vitro applications the data can easily be transferred to a computer where on- or off-line analysis can take place, such a high data rate is generally incompatible with the low-power requirements of an implant. Indeed, implanted systems have bandwidth-limited RF links and severe power budget and dissipation constraints (dissipation must be lower than 80 mW/cm² which corresponds to the chronic heat dissipation level considered the limit to prevent tissue damage).

Moreover, the recorded waveforms contain mostly noise data. As the typical firing rates of neurons are in the range of 10-50 per second per electrode and the duration of a spike signal is typically 1-3 ms, 85-99% of the samples represent only noise. Embedded real-time signal processing becomes then necessary to reduce the data flow while extracting the relevant information. Several spike detection algorithms have been proposed; these methods mostly operate on computers and their transcription into low-power hardware architectures is a major challenge.

A number of implantable system architectures are known from the prior art. However, all these systems require some level of human supervision, and therefore they do not allow fully autonomous recording of neural activity.

International Application WO 2006/003662 describes a neuronal recording system for automatic spike detection and alignment. The system comprises a processor which can be placed next to recording electrodes and provide for all stages of spike processing, stimulating neuronal tissues and wireless communications to a host computer. Some of the algorithms implemented by this processor are based on PCA (principal component analysis). These algorithms execute autonomously, but require off-line training and setting of computational parameters.

The paper by R. R. Harrison, “The design of integrated circuits to observe brain activity”, Proceedings of the IEEE 96: 1203-1216, July 2008 describes both an algorithm and an analog circuit for the automatic detection of spikes in neural recordings. The technique makes use of the statistical property of the normal distribution to determine the value of a discrimination threshold. The implementation is fully analog and is completely integrated in an ASIC. The detection operates successfully only for large spike signals (most likely intracellular) and suffers from its sensitivity to signal-to-noise ratio variations. Because of its entirely analog architecture, the technique also suffers from power dissipation issues. The main drawbacks of this method are its sensibility to signal to noise variations and its reliance on the analog performances of the comparator. The paper by Amir M Sodagar et al., “A fully integrated mixed-signal neural processor for implantable multichannel cortical recording”, IEEE Trans. Biomed. Eng., vol. 54, no. 6, pp. 1075-1088, June 2007 discloses a 64-channel neural processor for an implantable neural recording microsystem. This processor is capable of detecting neural spikes using programmable positive, negative or window thresholding, implementing a technique called “hard thresholding”. This system does not operate in real-time and is both supervised and not autonomous. Another issue associated with this system is the lack of precision of the discrimination technique which leads to poor performances in the classification of the spikes.

The paper by Z. Nenadic and J. W. Burdick, “Spike Detection Using the Continuous Wavelet Transform” IEEE Transactions on Biomedical Engineering, vol. 52, n°1, January 2005, pages 74-86, describes a method for detecting spike using the continuous wavelet transform. The signal is analyzed at several scales, and a binary test is performed for every sample and at every scale. Then, the individual decisions are combined by taking advantage of the temporal localization properties of wavelets. This algorithm is very heavy from a computational point of view (number of operations and required memory), because the signal is analyzed at several resolution levels and without decimation. This makes its implementation in implantable devices very difficult, if not impossible.

The paper by I. Obeid and D. Wolf “Evaluation of Spike-Detection Algorithms for a Brain-Machine Interface Application, IEEE Transactions on Biomedical Engineering, vol. 51, n°6, June 2004, pages 905-911, considers a plurality of simple spike detection methods, and in particular the “Non-linear Energy Operator” (NEO) algorithm, which seems giving the best results. However, threshold computation for this algorithm is still an unsolved problem.

Detected spikes need to be sorted or classified in order to extract useful information. Sorting consists in associating each individual spike to a specific neuron in the vicinity of a MEA implant. Indeed, the spikes issued from different neurons have different features, due to the different electrical path between each neuron and the nearest electrode of the array; see e.g. the review paper by M. S. Lewicki, “A review of methods for spike sorting: the detection and classification of neural action potentials”, Computation in Neural Systems, vol. 9, no. 4, pp. R53-R78, 1998.

In practice, performing real-time embedded spike sorting for a large number of channels is difficult, in particular due to low-power restrictions. Therefore, sorting is usually performed off-line, after the data has been collected: see for example the paper by A. Zviagintsevet al. “Algorithms and architectures for low power spike detection and alignment”, Journal of Neural Engineering, vol. 3, pp. 35-42, 2006. A standard approach in spike sorting consists in automatically determining features obtained from a principal component analysis (PCA) and only retaining the few principal components that catch most of the signal variance: see the paper by P. M. Horton et al., “Spike sorting based upon machine learning algorithms (soma)”, Journal of Neuroscience Methods, vol. 160, no. 1, pp. 52-68, 2007.

Computation of the projection matrix coefficients for the PCA is computationally heavy. Hence it is usually performed off-line. These coefficients are then uploaded into an embedded process for on-line real time processing of signals. The offline computation of the coefficients makes any system using it not autonomous and non-adaptative unless the computation is frequently repeated to account for any evolution in the signal characteristics and which overburdens the system operation.

Other sorting methods operate directly on spike shape features. Indeed, the main characteristics of spikes are known: they have a rather smooth biphasic or triphasic waveform with a finite temporal duration. It is then straightforward to consider a few spike shape features for sorting. Most common features such as spike minimum and/or maximum, width and peak-to-peak amplitude are easily implemented. Performances using shape features are known to compare well with PCA-based methods; see D. J. Sebald, A. Branner, “Automatic Spike Sorting For Real-time Applications”, Proceedings of the 3rd International IEEE EMBS Conference on Neural Engineering, pp. 670-674, Kohala Coast, Hi., USA, May 2-5, 2007.

Sorting techniques allow analyzing data in order to reveal clusters that are relevant for classifying spikes. “Clustering” refers to the operation of automatically determining the cluster boundaries. Many methods for spike sorting have been proposed such as Bayesian clustering (see the above-referenced paper by M. S. Lewicki), K-means (S. Takahashi, Y. Anzai, Y. Sakurai, “Automatic sorting for multi-neuronal activity recorded with tetrodes in the presence of overlapping spikes”, Journal of Neurophysiology, vol. 89, pp. 2245-2258, 2003) or Gaussian mixture models (C. Pouzat, O. Mazor, G. Laurent, “Using noise signature to optimize spike-sorting and to assess neuronal classification quality”, Journal of Neuroscience Methods, vol. 122, pp. 43-57, 2002).

These classifiers yield excellent results but they are hardly suitable for an embedded implementation. Furthermore, they usually need an off-line training process before the decision parameters be downloaded to the data analysis processor. Moreover, these clustering techniques will usually fail if the neural activity changes with time.

There is currently a need for algorithms and architectures suitable to perform spike sorting/clustering in an embedded signal processing device associated to the MEA, because this would allow a much greater reduction in the data volume to be transmitted outside the body than spike detection and extraction alone.

As an example, let us consider a 64-electrode MEA with a 12.8 kSps sampling frequency and a 12-bit digital representation; as discussed above, the data rate of the “raw” signal is 9.6 Mbit/s. Spike detection and extraction allows a first reduction of the data rate, e.g. by a factor of about 16, assuming a spike rate of 25 useful pulses per second and per electrode, and by extracting 32 samples for each spikes. After spike sorting, each spike is simply represented by a neuron identifier (4 bits) and a time stamp (16 bits), i.e. 500 bit/s/electrode or 31.25 kbit/s for the whole 64-electrode system. The overall data compression rate is 314, to be compared to the compression rate of 16 obtainable through spike extraction alone.

The invention aims at providing methods and devices for performing fully automated, unsupervised and embedded detection and/or classification of neural spikes. Medical implants based on the invention are expected to be suitable to closed-loop applications, in which real-time detection and information processing are followed by adequate and specific electro-stimulation for diseases like Parkinson's and epilepsy.

More precisely, according to claim 1, a first object of the invention is a method for estimating a level of noise affecting a sampled and digitized pulse signal, such as a neural action potential signal, comprising the steps of: truncating the values of digitized samples exceeding a threshold value; collecting truncated samples over a time window, and determining statistical frequencies of the corresponding values; identifying or estimating a maximum statistical frequency of the collected samples; identifying a truncated sample value whose statistical frequency is nearest to a predetermined fraction of said maximum statistical frequency; and estimating a noise level from the thus-identified truncated sample value.

The method of the invention is particularly simple from a computational point of view, and is well-suited for low-complexity and low-power hardware implementation.

Particular embodiments of such a method are addressed by dependent claims 2 to 7.

A second object of the invention, according to claim 8, is a method for detecting signal pulses, such as neural spikes, the method comprising the steps of: sampling and digitizing an input signal containing the pulses to be detected; estimating a level of a noise affecting said input signal by a method according to any of the preceding claims; determining a threshold level depending on the estimated noise level; and deciding that a pulse has been detected whenever the input signal, or an absolute value thereof, exceeds said threshold level.

As the threshold is determined on the basis of the estimated noise level, this method is fully autonomous and does not require any supervised training. Moreover, the threshold can automatically adapt to a time-varying noise level.

Particular embodiments of such a method are addressed by dependent claims 9 to 12.

A third object of the invention, according to claim 13, is a device for performing such a method, comprising: an input port for receiving a sampled and digitized signal; means for truncating at least one most significant bit of the received signal samples; a digital memory comprising at least 2^{k} memory locations, k being the number of bits of the truncated signal samples, each memory location being identified by a unique address corresponding to a possible value of said truncated samples; means for initializing said digital memory; means for incrementing a value stored in the memory locations whose address correspond to a possible truncated sample value upon reception of a corresponding sample; means for determining or estimating a maximum value stored within said digital memory; means for determining the address of a memory location storing a value which is nearest to a predetermined fraction of said maximum value; and means for computing a level of a noise affecting the input signal from the thus-determined address.

Such a device constitutes an advantageous hardware implementation of the methods of claims 1 to 12.

A fourth object of the invention, according to claim 14, is an electronic circuit comprising: at least an input port for an analog input signal comprising signal pulses and noise; means for sampling said analog input signal and converting it to digital format; a digital band-pass filter matched to an expected shape of pulses contained within said signal, connected for filtering the digitized signal; a device as described above, receiving as its input port the filtered digitized signal; means for computing a threshold level as a function of an estimated noise level outputted by said device; comparator means of detecting a pulse whenever the digitized input signal crosses said threshold level; and means extracting a set of samples of the digitized and filtered signal, having a predetermined size, corresponding to each detected pulse.

A fifth object of the invention, according to claim 15, is an implantable neurobiological recording system comprising: a multi-electrode array for acquiring neural action potential signals; an electronic circuit as described above, receiving at its input port(s) signals acquired by said multi-electrode array; and means for transmitting signals outputted by said electronic circuit to non-implantable external devices. Thanks to its inventive architecture, such a neurobiological recording system is able to perform unsupervised and real time spike identification, while complying with the stringent power and size requirements for implantable device.

A sixth object of the invention, according to claim 16, is a method for classifying (i.e. sorting and clustering) pulse signals comprising the steps of: quantifying a set of predetermined features of a pulse signal to be classified; representing said pulse signal as a point in a feature space containing a dynamically updated set of clusters of points, a subset of said clusters being considered as significant; including the point representing said pulse signal in a nearest cluster—either significant or not—according to a predetermined metric, or create a new non-significant cluster centered on said point, if its distance from any existing cluster exceeds a threshold; and classify this same point as being associated to a nearest significant cluster according to said metric; and updating the set of clusters taking into account the thus-classified signal.

Such a method is particularly well-suited for performing embedded on-line (real-time) adaptive and unsupervised classification of spikes.

Particular embodiments of such a method are addressed by dependent claims 17 to 22.

A seventh object of the invention, according to claim 23, is a method of processing a sampled and digitized pulse signal comprising detecting pulses by a method according to any of claims 8 to 11; and classifying the detected pulses by a method according to any of claims 16 to 22.

An eighth object of the invention, according to claim 24, is an electronic circuit as described above, further comprising: data processing means for classifying the detected pulses by a method as described above; and means of outputting classification results and events affecting significant clusters.

A ninth object of the invention, according to claim 25, is an implantable neurobiological recording system comprising: a multi-electrode array for acquiring neural action potential signals; an electronic circuit as described above, receiving at its input port(s) signals acquired by said multi-electrode array; and means for transmitting signals representative of classification results and events affecting significant clusters, outputted by said electronic circuit, to non-implantable external devices.

Thanks to its inventive architecture, such a neurobiological recording system is able to achieve very efficient data rate compression, while complying with the stringent power and size requirements for implantable device.

As discussed above, the methods and devices of the invention are directed in particular to embedded real-time processing of neural signals. However, they can also find other applications, e.g. for processing signals generated by different kinds of detectors, such as radiation detectors. Therefore the invention is not limited to neural applications.

Additional features and advantages of the present invention will become apparent from the subsequent description, taken in conjunction with the accompanying drawings, which show:

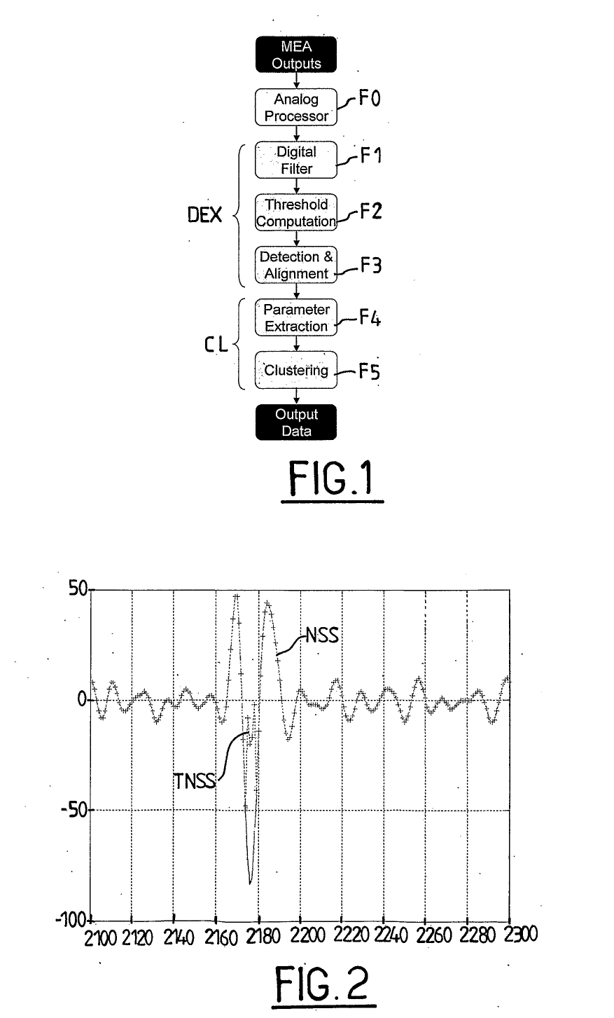

FIG. 1, a general, high-level block diagram of a neural signal processing algorithm to which the invention can be applied;

FIG. 2, a neural spike signal (continuous line) and its truncated counterpart (dotted line) used for noise level estimation;

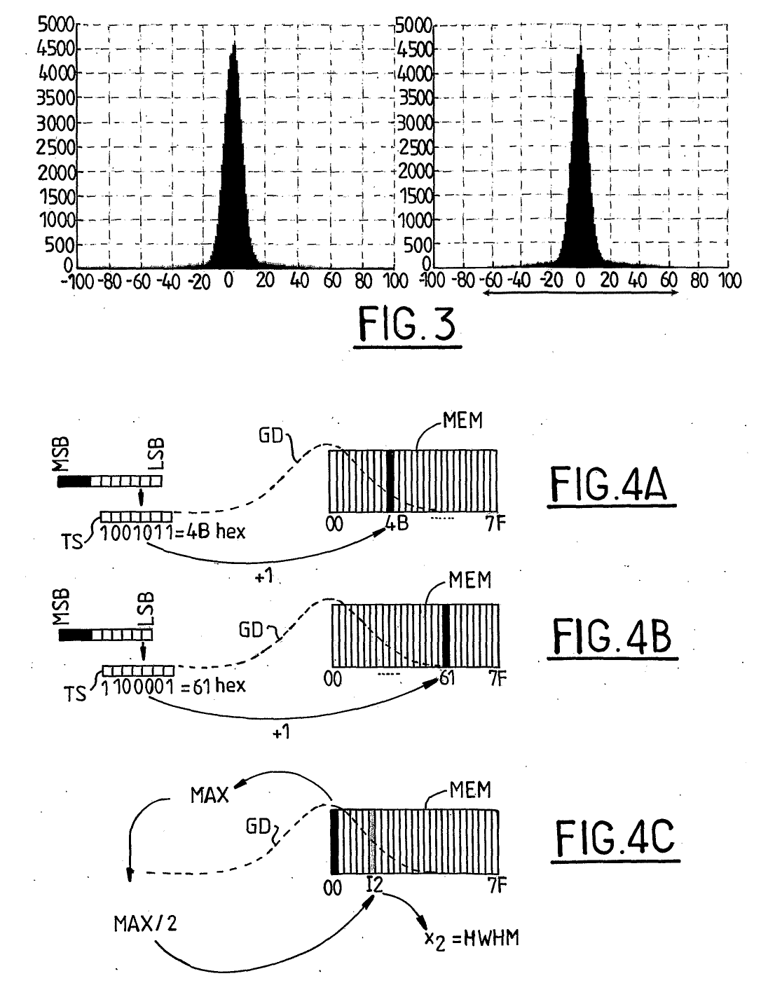

FIG. 3, the statistical distributions of signal samples (left panel) and of their truncated counterparts (right panel);

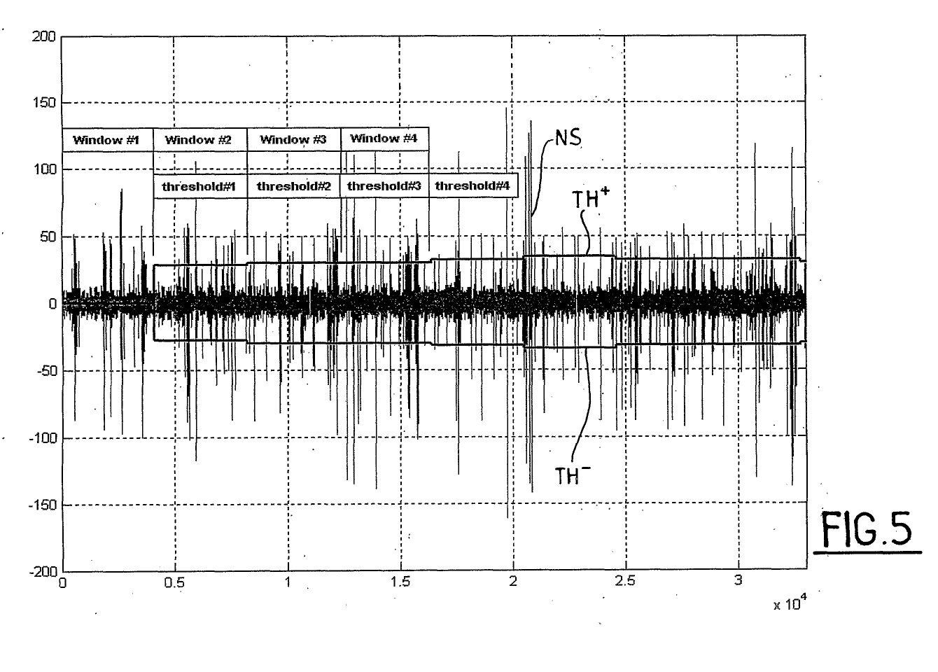

FIGS. 4A, 4B and 4C, different steps of a method according to an embodiment of the invention for determining the noise level of a pulse signal;

FIG. 5, a plot illustrating continuous real time determination of a discrimination threshold for spike detection according to an embodiment of the invention;

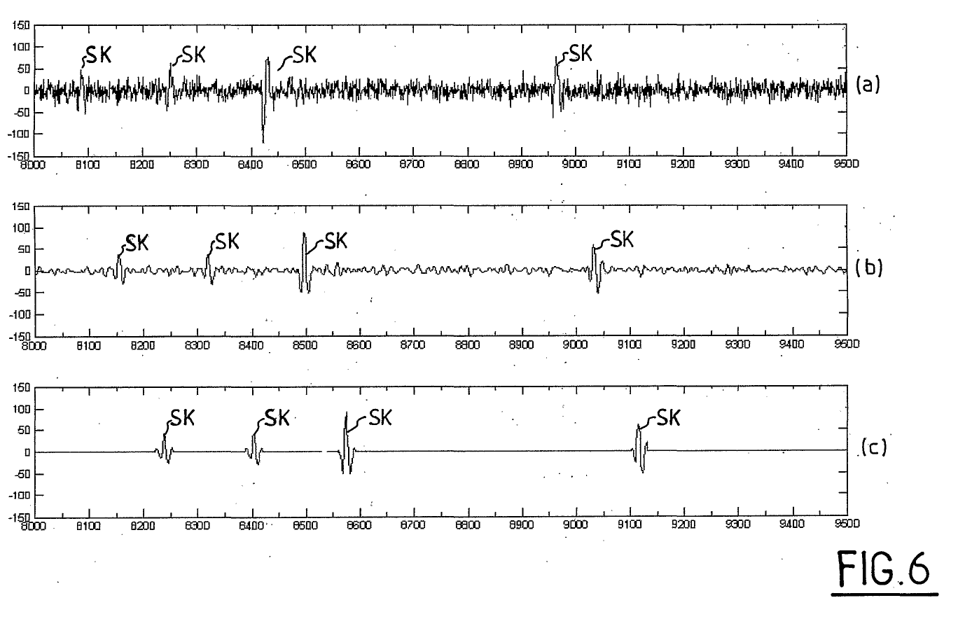

FIG. 6, three temporal plots of a raw MEA signal, of a filtered version of the same signal and of extracted spikes, respectively;

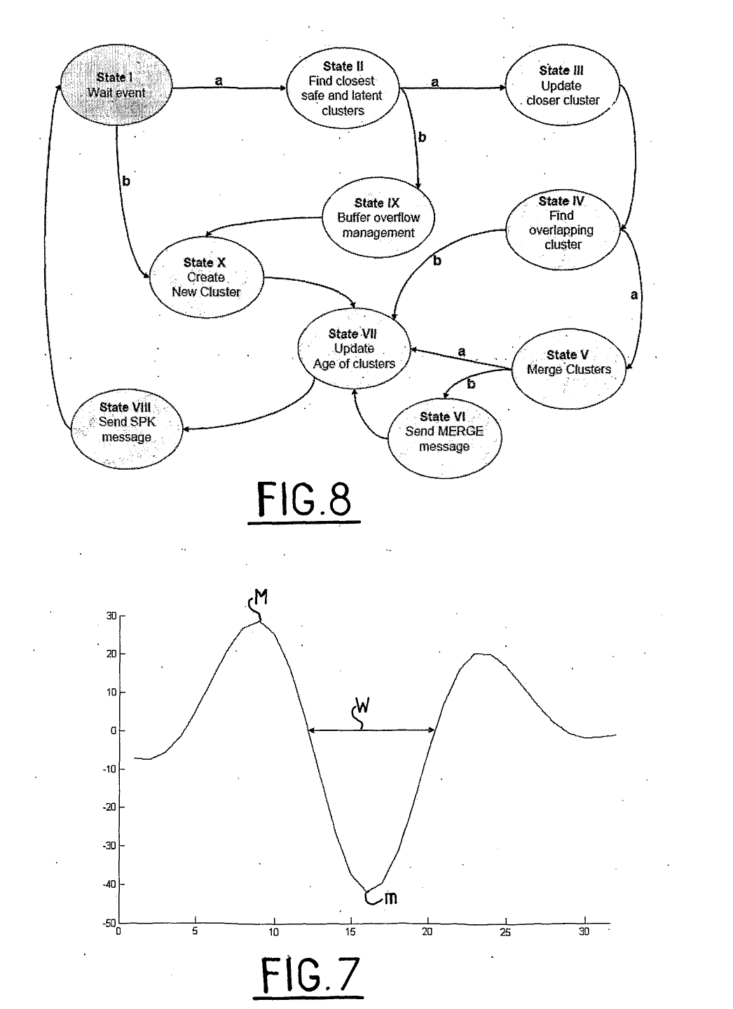

FIG. 7, the plot of an extracted spike, illustrating the features used for classification;

FIG. 8 a state machine representation of the spike classification algorithm according to an embodiment of the invention; and

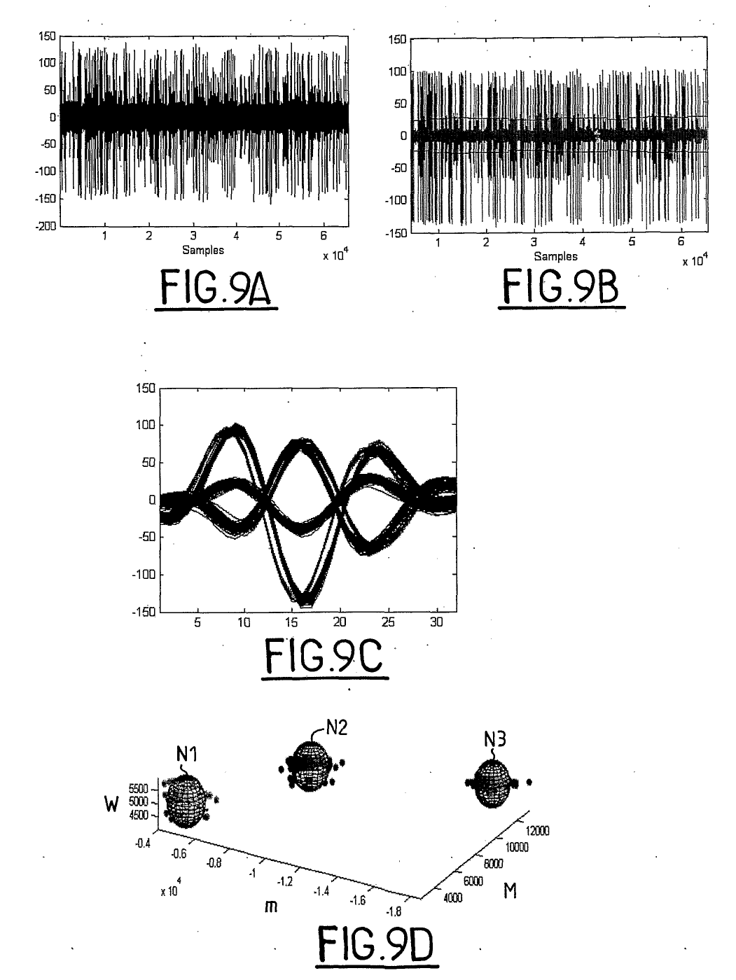

FIGS. 9A, 9B, 9C and 9D, plots illustrating a simulation performed for validating the spike extraction and classification algorithms of the invention.

In the plots of FIGS. 2, 5, 6, 7, 9A and 9B, the X-axis represents samples and the Y-axis signal amplitude, in arbitrary units. In the histograms of FIG. 3, the X-axis represents amplitude bins and the Y-axis the corresponding numbers of counts.

Real-time embedded signal processing is a challenging yet mandatory step in the development of multi-electrode array instrumentation; as discussed above, the data flow generated by large electrode arrays must be reduced to envision compact data acquisition systems with wireless transmission for body implantation.

FIG. 1 represents the general block diagram of an algorithm for processing neural spike signals issued by a multi-electrode array MEA, the algorithm comprising the following basic functions:
bandwidth reduction for selective band amplification and noise reduction, F1;
discrimination threshold computation, F2;
detection, extraction and alignment of neurobiological spike signals, F3;
dimension reduction, obtained with a space shape features or Principal Component Analysis, F4; and
spike clustering; F5.

Bloc F0 refers to the preliminary operations of analog preamplification, sampling and analog-to-digital conversion.

Functions F1-F3 constitute the spike detection and extraction (DEX) step, while functions F4 and F5 constitute the spike classification step (CL).

A goal of the invention is to make possible to implement all the processing functions F1-F5 on a single, low-power circuit (preferably an application-specific digital integrated circuit) interfaced with an analog processor implementing the preliminary operation F0.

Selective bandwidth filtering (function F1 on FIG. 1) is used to improve the signal-to-noise ratio SNR. Spike signals are known to have a typical bandwidth comprised between 300 Hz and 3 kHz, and a stereotypical bipolar shape; therefore, matched filtering techniques can be used.

For carrying-out the invention, it is particularly advantageous to use the quadratic spline matched filter described in the paper by Ricardo Escolá et al., “Wavelet-based scale-dependent detection of neurological action potentials”, Proceedings of the 29th Annual International Conference of the IEEE EMBS, Lyon, France, Aug. 23-26, 2007. This is a band-pass filter centered on 1 kHz, selectively amplifying the total signal in the spike frequency band. The resulting attenuation in the low frequency band (below 300 Hz) provides rejection of both DC components and Local Field Potentials (LFP). The one in the high frequency band (above 3 kHz) limits the overall bandwidth of the total signal, effectively reducing the noise band. The term “total signal” refers to the sum of the noise and of the action potentials (spikes). Because of the filter selectivity and of the resulting bandwidth reduction, the SNR is increased. Use of a Finite Impulse Response (FIR) filter with 14 power-of-two coefficients makes its hardware implementation simple and low-power. However, other kind of filters can also be used.

In order to discriminate significant spikes from noise, the filtered signal undergoes thresholding (F3), and fixation of the discrimination threshold turns out to be a critical operation.

It is commonly admitted that the noise distribution in extracellular recordings is essentially white and follows an almost-Gaussian distribution. It is known that, under these conditions, setting the discrimination threshold between three and five times the value of the noise distribution standard deviation allows pulse detection with a low probability of false positives; setting the threshold higher further reduce the false positive probability, while also lowering the overall detection sensitivity. See the above-cited paper by I. Obeid and P. Wolf.

A robust estimate of the standard deviation of the white Gaussian noise distribution is therefore required for threshold value determination.

The standard deviation a of a finite set of samples can be computed based on its mathematical expression:

$$σ = \frac{1}{N - 1} \sqrt{{\sum }_{i = 1}^{N} {( {x}_{i} - \overline{x} )}^{2}}$$

where xᵢ is the value of the i-th sample and x̅ is the mean value:

$$\overline{x} = \frac{1}{N} {\sum }_{i = 1}^{N} {x}_{i}$$

It should be noted that the equations above allow calculation of the standard deviation of the total signal, and not that of noise alone. However, it can be reasonably assumed that the contribution of signal pulses is negligible compared to that of noise.

Direct evaluation of the standard deviation requires four distinct computational steps; the first one consists in evaluating the simple mean of the sample population; the second involves the use of the square function, whose hardware implementation is complex; the third is a summation followed by a root mean square operation, whose hardware implementation is also complex; the fourth and final step is a division, which can be as simple as a bit shift provided that N is chosen to be a power of 2.

This method of computing the standard deviation computation suffers from two main drawbacks. First of all, it is sensitive to the mean variation of the distribution and to outliers, which artificially widen the distribution and overestimating the true value of the standard deviation. Moreover, the complexity of both the square and the root mean square operation makes the implementation of this solution as a simple and low power embedded architecture very unlikely.

Another possibility, disclosed by the above-cited papers by Escola' and Nenadic, consist in computing the Median Absolute Deviation (MAD), defined as:

MAD=med{|xᵢ− x̅|, i=1 . . . n}

For a Gaussian distribution, MAD is linked to the standard deviation by MAD=0.6745×σ.

This solution has a lower complexity than the direct standard deviation computation presented in the previous section and is also robust to outliers. However, its complexity is still too great for implementation in a simple and low power embedded architecture.

An object of the invention is a new method for determining the level of a noise affecting a neural signal. This method is very simple to implement in a low-power digital architecture.

The idea at the basis of this method is to determine the Full Width at Half Maximum (FWHM) value of a Gaussian distribution, rather than its standard deviation, which is generally used as a measure of the noise level. Indeed, for a Gaussian distribution, the standard deviation is approximately equal to 2.35 times the FWHM.

The method of the invention comprises the following steps.

First of all, signal samples are truncated by dropping one or more Most Significant Bits (MSB). The reason for this is that noise only influences the least significant bits (LSB) of the digital values, while the MSBs are different from zero only during neural spikes and therefore are not useful for estimating the noise level. Of course, the number of LSBs must be chosen so as to accommodate a large range of peak-to-peak noise values. Samples corresponding to truncated spikes will alter the precision of the threshold estimate, but not significantly because of their low occurrence rate. Moreover, the threshold value is computed as an integer value and the round-off effect introduces a larger error than the truncation.

FIG. 2 represents a band-pass filtered neural spike signal NSS (continuous line) and its truncated counterpart TNSS (dotted line). In this case, complement to 2 encoding of samples has been used.

On FIG. 3, the left panel shows the distribution of samples of a MEA signal quantized on 10 bits, and the right panel the distribution of the corresponding truncated samples (six LSBs plus the sign bit). It can be seen that the distributions, which are almost equal, are not truly Gaussian as they have long tails, due to the spikes. The double arrow below the right panel of FIG. 3 represents the dynamic range of truncated samples.

The truncated samples, comprising k bits, can be seen as addresses of individual locations of a 2^{k}-location digital memory MEM, previously initialized to zero. Whenever a truncated sample TS is received, the content of the corresponding memory location is incremented by one unit. This way, a signal histogram is built in real time. For example, FIGS. 4A and 4B represent the increment of the content of the memory locations whose addresses (in hexadecimal format) are 4B and 61, respectively. The dotted line GD superposed to the memory MEM represents the Gaussian distribution of the samples, which will be approximated by the histogram.

As the distribution of Gaussian noise is symmetric and zero-mean, it is possible to take into account positive (or negative) samples only, and to build a histogram of the half-distribution. Generalization to the non-zero-mean case is trivial.

When a sufficient number of samples has been acquired, the maximum value MAX of the histogram is determined. As the Gaussian distribution is symmetric, this maximum value should normally correspond to the value stocked in the location whose address is 00. In order to reduce the statistical errors, it is possible to average the values stocked in a small number of low-address memory locations (e.g. 00-03). Then the digital memory is scanned for determining the location whose content is nearest to MAX/2. The address x₂ of this location is the approximated HWHM—Half Width at Half Maximum—of the signal distribution. This step is illustrated on FIG. 4C.

A detailed analysis shows that the number of operations required to compute the HWHM according to the method of the invention is significantly smaller than that required for computing the MAD according to the prior art. This is particularly true when the signal mean is different from zero: in this case, the MAD algorithm requires computation of the signal mean, which is not necessary for carrying out the method of the invention.

A suitable discrimination threshold θ can be obtained by multiplying x₂ by a predetermined constant κ, typically comprised between 3 and 5. It is particularly convenient to take κ=4, because multiplication by four can be very simply performed by bit shifting. Moreover, θ=4×HWHM=2×FWHM 4.7σ, which is a good tradeoff between sensitivity and rejection of false positives.

Using only a fraction of the dynamic range might lead to errors because of baseline fluctuations; however, those occurring outside the digital filter bandwidth are removed. To account for the fluctuations of the baseline within the filter bandwidth, the computation of the threshold is not performed once, but continuously on consecutive sets of samples. As a consequence the threshold value is computed in real-time every few hundreds of milliseconds, which is compatible with computation latency in BCI (Brain-Computer Interface) applications. The refreshing rate of the threshold value (every few hundreds of milliseconds) is faster than the rate of change of the noise standard deviation making the threshold variation from window to window negligible. This result is used in the digital architecture implementation of the algorithm, where the value computed over one window is effectively used to discriminate the data of the next set of samples while the new threshold value is computed. This is illustrated on FIG. 5, where:
the parallel segments TH+, TH− superposed to the plot of the noisy signal NS represent the (identical, in absolute value) positive and negative thresholds;
it can be seen that the first threshold, “threshold #1”, computed during the first time window, “Window #1” is applied to the signal during the second window “Window #2”, and so on.

Within each window, a spike is detected whenever the absolute value of the signal exceeds the threshold value. Upon detection, a set of samples, whose duration approximately corresponds to that of a spike, is extracted from the signal. Typically, the duration of a spike is of the order of 2.5 ms; assuming a sampling rate of 12.8 kSpS, the extracted set can comprise 32 samples. The extraction procedure is performed so as to provide a 32-element sample vector aligned on the absolute maximum of the extracted signal; this can be obtained, e.g. by detecting the time at which the spike signal exceeds a threshold, and by extracting a predetermined number of samples before and after said time. The accuracy of the extraction is particularly important in cases where the PCA method is used as the next step. The extraction rests on data pipelining methods coupled to the over-the-threshold detection signal.

The extraction routine can also provide a veto signal hindering the triggering of the comparator during a suitable “refractory period”.

The upper panel of FIG. 6 shows a temporal plot of the “raw” MEA signal (after analog preamplification and filtering). The median panel shows a plot of the same signal after band-pass filtering by a matched wavelet filter: it can be seen that there is a very significant improvement of the SNR, which makes spike detection much easier. Finally, the lower panel of FIG. 6 represents the extracted spikes SK, zero-padded for illustration purposes.

The temporal shift between the plots of FIG. 6 is an artifact reflecting the delay induced by real-time computation.

The extracted spikes can then be classified in order to associate each individual spike to the neuron from which it originates. On-line classification can be performed by a method according to the invention, and which will be described below.

Prior to clustering proper, it is necessary to perform dimension reduction of the extracted spikes. Dimension reduction consists in replacing the full waveform of the spike (represented e.g. by 32 samples) by a much smaller number of significant features. For example, as represented on FIG. 7, three features can be used: maximum (M), minimum (m) and width of the main lobe (W).

Normalization is necessary for the subsequent classification step, to ensure that the variation ranges of all the different features have similar orders of magnitude. In an advantageous embodiment of the invention, power-of-two coefficients are used to obtain similar orders of magnitude for the three parameters. Those power-of-two coefficients make the normalization process easy to implement in hardware, because dividing or multiplying a number in binary format by a 2^{k}, with k integer, only requires a shift of k positions of the bits of said number. Those coefficients are also application-specific and must be computed for each experiment, based on characteristics of the monitored signal. Advantageously, they can be automatically extracted from threshold computation.

It should be understood that any number and any combination of parameters can be used to characterized and differentiate spikes. There is however a trade-off between the number of parameters used and the precision of the classification: indeed, use of too many parameters hinders the classification. The various combinations and the numbers of selected parameters must be evaluated in respect to complexity and performance in order to reach a compromise. A non-exhaustive list of parameters comprises:
distance between the minimum and the maximum; area of the different lobes; energy of the signal;
number of times the signal is over the threshold; and
peak-to-peak amplitude.

After dimension reduction, each spike is represented by a point in a N-dimensional space, N being the number of features considered (N=3 in the present example). As spikes emitted by a same neuron are similar to each other and different from spikes emitted by other neurons, spike-representing points tend to form clusters in the N-dimensional feature space. The problem of classification consists in identifying the boundaries of said clusters, and therefore attributing univocally each spike to a single cluster (or labeling a spike as an “outlier” to be discarded). For the present application, classification has to be performed “on-line”, i.e. while spikes continue to be acquired, and without supervision. Moreover, the classification algorithm needs to be simple enough to allow its implementation in an implantable electronic circuit.

The classification method of the invention complies with these stringent requirements. This method is based on progressive update of a clustering model upon the arrival of each new event (i.e. spike), which allows tracking changes in the clusters position in the feature space. This is important, because the MEA can move slightly within the brain, and this induces a change of the shape of the spikes.

Each cluster j has the shape of a hypersphere in the N-dimensional feature space, and can be compactly described by four parameters:
Location of its center: Ωⱼ;
Radius: rⱼ;
Number of events contained within the cluster: Nⱼ; and
Cluster age: Aⱼ.

A distinction is made between “significant” or “safe” clusters, which are supposed to correspond to actual neurons, and “latent” or “non-significant” clusters. Only “significant” clusters are visible outside the classification algorithm. Concretely, a cluster j is considered as “safe” or significant if it contains a number of events superior to a predetermined threshold (Nⱼ≧N*); otherwise it is considered as latent. The threshold N* is different for each application and depends on the duration of the recording.

It should be understood that more than four parameter, possibly different from those listed above, could be used, such as the median for the center location, or the farthest element, etc.

The main steps of the classification method of the invention are:

(a) Determination of the Distance Between an Incoming Event and the Existing Clusters:

Upon the arrival of a new spike, the selected features are used to calculate their distances from the boundaries of all clusters.

dⱼ=d(s,Ωⱼ)−rⱼ

where the index j identifies the cluster, s represents the new event in feature space, Ωⱼ, rⱼ are the center and the radius of the cluster j and d(.) is a suitable metrics, such as a normalized “Manhattan” distance, defined as:

$$d ( x , y ) = \frac{1}{\sqrt{N}} {\sum }_{i = 1}^{N} \vert {x}_{i} - {y}_{i} \vert$$

The “Manhattan” distance is a very useful metric for comparing distances between pair of points, because it generally yields similar results to the Euclidean distance without using the square root and power operations which are too burdensome to integrate into an embedded system. However, it is not such a good estimator of absolute distance and its performances degrade quickly when the number of dimensions increases. Normalization, taking into account the number of dimensions, reduces the corresponding error.

(b) Cluster Creation or Cluster Affectation

If the event lies within the hyper-volume defined by a cluster (safe of not), it is added to this cluster; otherwise, a new cluster is created, whose center coincides with the point representing the event. In both cases, the event is also associated to the closest “safe” cluster, and this association is the only information which is communicated to the “outer world” during this step.

(c) Cluster Update

The three parameters Ωⱼ, rⱼ and Nⱼ of the nearest cluster (“safe” or not) to which a new event has been affected are updated recursively:

$$∀ {N}_{j} < {N}^{*} , {Ω}_{j} = \frac{{N}_{j} {Ω}_{j} + s}{{N}_{j} + 1}$$

else Ωⱼ=Ωⱼ

Nⱼ=Nⱼ+1

rⱼ=constant=σ, σ being the noise standard deviation calculated during spike detection.

The age Aⱼ of the cluster remains unchanged at this step.

(d) Cluster Merge

When the distance between two clusters a and b is less than the length of both radii (or is less than a threshold distance defined as a function of one or both the radii), the two clusters are merged into a new one. By convention, the new cluster retains the identifier of cluster a, while cluster b is erased. The merging condition can be written:

d(Ωₐ,Ω_{b})<max(rₐ,r_{b})

The parameters identifying the “merging” cluster are determined as follows:

$${N}_{M} = {N}_{S} + {N}_{W}$$

$${Ω}_{M} = \frac{{N}_{S} {Ω}_{S} + {N}_{W} {Ω}_{W}}{{N}_{S} + {N}_{W}}$$

$${r}_{M} = {r}_{S} + d ( {Ω}_{S} , {Ω}_{M} )$$

In the equations above, the index S and W means “strong” and “weak” respectively, the “strong” cluster being that with the largest number of elements.

(e) Age Update

At the end of the processing of an event, whether a new cluster was created or two clusters merged, the age update procedure takes place. Let i be the index of the cluster whose age has to be updated, then the procedure is as follows:

∀k≠i Aₖ=Aₖ+1

Aᵢ=0

In the case of cluster creation, the index i refers to the index of the new created cluster. In the case of cluster merge, the index i refers to the index of the closest safe cluster.

As it can be easily understood, the number of clusters cannot increase without limits. Therefore, when the number of cluster exceeds a threshold, the cluster with a lowest priority is suppressed.

Age is a measure of the activity—or, more precisely, of the inactivity—of a cluster: as the age of a cluster is reset to zero when a new event is added to it while the age of the other clusters increases, “old” clusters are the less active. According to the invention, the cluster priority level determined according to the number of events Nⱼ and the age Nⱼ: “young” clusters (low Aⱼ) with many events (high Nⱼ) have high priority, while “old” clusters with few events have low priority. For example, priority can be computed as Nⱼ−Aⱼ or, more generally, as αNⱼ−Aⱼ, a being a weighting coefficient.

It should be noted that the radius of the cluster does not change due to the arrival of new events (it remains equal to 6, i.e. to the noise standard deviation), but only due to merging. Optionally, variations of the noise level observed by the spike detection algorithm can be used to update in real or semi-real time the radius of the clusters, independently from merging.

Only changes regarding safe clusters are communicated to the external world by two kinds of messages: SPK (spike) and MERGE.

A SPK message is generated whenever an event (a spike) is received. It comprises a datagram containing the time-stamp of the spike and the identification value (k) of the (safe) cluster to which the event has been associated.

When two “safe” clusters merge (and only in this case), a MERGE message is emitted, too, containing the identification value k of the new cluster and those of its parents.

At the beginning, there are no clusters. “Latent” clusters are created, centered on the first detected events; then, these latent clusters grow and merge with the arrival of new events, and some of them eventually become “safe”. As a consequence, there is an initialization period during which no significant information is outputted by the algorithm.

FIG. 8 represents the classification algorithm in the form of a state machine comprising 10 states:
STATE I. Wait for a new event. The transition occurs when an event sᵢ arrives.

a. If the cluster space is non-empty, go to STATE II.

b. If the cluster space is empty, go to STATE X.
STATE II. Find the closest cluster: Clusterₘ and the closest safe (“safest”) cluster: Clusterₖ. The transition occurs when all existing clusters are evaluated.

a. If sᵢ falls inside the radius of the closest cluster (i.e. Clusterₘ exists), go to STATE III.

b. If sᵢ falls outside the radius of the closest cluster (i.e. there is no Clusterₘ), go to STATE IX.
STATE III. Update Clusterₘ with the new event. The transition occurs when Ωₘ, rₘ and Nₘ have been recalculated. Go to STATE IV.
STATE IV. Given a Clusterₘ, find and overlapping Clusterⱼ satisfying:

∀j≠m,d(Ωₘ,Ωⱼ)<max(rₘ,rⱼ) and

d(Ωₘ,Ωⱼ) is minimal

The transition occurs when all clusters have been evaluated against Clusterₘ.

a. If there is an overlapping Clusterⱼ, go to STATE V.

b. If there are no overlapping clusters, go to STATE VII.
STATE V. Merge overlapping Clusterₘ and Clusterⱼ into Clusterₙ. The transition occurs when the merging process is completed.

a. If m≠k, then the sub-critical Clusterₘ has either being merged with another sub-critical cluster, or absorbed by a safe one. In either case the merging should be transparent to the outer world and no MERGE message should be sent because the classified cluster identifier remains the same. Go directly to STATE VII.

b. If m=k, then the classification identifier may have changed in the merging scheme (because the smaller index is always kept) and no longer be valid. Merging must be reported in order to keep the integrity of the classification. Go to STATE VI. If there is no merging, skip to STATE VII.
STATE VI. Send MERGE message. Transition occurs when the message is sent correctly. Then, goes to state VII.
STATE VII. Update ages of all clusters. Go to STATE VIII.
STATE VIII. Send SPK message. Transition occurs when the message is sent correctly. Return to STATE I.
STATE IX. Check that there is enough memory for a new cluster. If not, delete the cluster with the smallest priority (defined as a function of element number and/or age, as discussed above). The transition occurs when there is enough memory for a new cluster. Go to STATE X.
STATE X. Create a new Clusterⱼ from event sᵢ. Index j must be fetched from all available identifiers (it should be the smallest). The transition occurs when Clusterⱼ has been initialized. Go to STATE VII.

The spike detection and classification algorithms have been tested on a simulated dataset comprising 286 spikes coming from three different neurons (95 spikes for the first one, 95 for the second one and finally 96 for the last one). FIG. 9A shows a temporal plot of this simulated dataset. A discrimination threshold is determined in real time, according to the invention (FIG. 9B, corresponding to FIG. 5), and pulses are extracted. FIG. 9C represents the 286 extracted spikes, aligned in order to make their maxima coincide. On FIG. 9D, the spikes are represented as points in the M-m-W three-dimensional feature space; clusters N1, N2 and N3 associated to the three neurons are represented as spheres. Each simulated spike has been correctly associated to the cluster corresponding to the neuron from which it originates.

The signal processing algorithms of the invention, including wavelet-based matched filtering, can be translated into a hardware description language, such as VHDL, for performing logical simulations. Simulations have been performed in a Xilinx environment as ISE and Modelsim, by assuming implementation on Xilinx Virtex-5 FPGA (Field-Programmable Gate Array). The estimated dynamic power is:

16.9 μW per electrode for the detection algorithm (including noise level estimation);

0.05 μW per electrode for the space reduction algorithm; and

0.85 μW per electrode for the clustering algorithm;

i.e. a total power dissipation of 17.8 μW per electrode. The total power dissipation for a matrix with 1024 electrodes is 18.2 mW. Assuming a 50 mm² chip (5 mm² chip for 100 electrodes), a ratio of about 36.4 mW/cm² is obtained, which is well below the 80 mW/cm² chronic heat dissipation level considered to prevent tissue damage. This confirms that the proposed methods can actually be implemented in an implantable device.

1-25. (canceled)

26. A method for estimating a level of noise affecting a sampled and digitized pulse signal, comprising:
(a) truncating values of digitized samples exceeding a threshold value;
(b) collecting truncated samples over a time window, and determining statistical frequencies of the corresponding values;
(c) identifying or estimating a maximum statistical frequency of the collected samples;
(d) identifying a truncated sample value whose statistical frequency is nearest to a predetermined fraction of the maximum statistical frequency; and
(e) estimating a noise level from the thus-identified truncated sample value.

27. A method according to claim 26, wherein the truncating the values of digitized samples corresponding to signal pulses is performed by dropping one or more most significant bits of the digitized samples.

28. A method according to claim 26, wherein the threshold value is chosen such that only samples corresponding to signal pulses are truncated.

29. A method according to claim 26, wherein the noise-affected signal has zero mean, and wherein only signal samples having a predetermined sign are used for noise-level estimation.

30. A method according to claim 29, wherein the maximum statistical frequency is estimated to be equal to the statistical frequency of the zero value of the samples.

31. A method according to claim 26, wherein the estimating a noise level comprises multiplying the identified truncated sample value by a predetermined coefficient.

32. A method according to claim 31, wherein the predetermined coefficient is between 3 and 5, and wherein the statistical frequency of the identified truncated sample is approximately equal to half the maximum value.

33. A method for detecting signal pulses, comprising:
sampling and digitizing an input signal containing the pulses to be detected;
estimating a level of a noise affecting the input signal by a method according to claim 26;
determining a threshold level depending on the estimated noise level; and
deciding that a pulse has been detected whenever the input signal, or an absolute value thereof, exceeds the threshold level.

34. A method according to claim 33, further comprising extracting a set of samples of the input signal, having a predetermined size, corresponding to each detected pulse.

35. A method according to claim 33, further comprising a preliminary band-pass filtering of the input signal by using a filter matched to an expected shape of the signal pulses.

36. A method according to claim 33, further comprising subdividing the input signal in a series of temporal windows, and wherein the detecting a pulse is performed by using a threshold level determined on the basis of signal samples belonging to a previous temporal window.

37. Use of a method according to claim 26 for processing neural action potential signals.

38. A device for performing a method according to claim 26, comprising:
an input port for receiving a sampled and digitized signal;
means for truncating at least one most significant bit of the received signal samples;
a digital memory comprising at least 2^{k} memory locations, k being the number of bits of the truncated signal samples, each memory location being identified by a unique address corresponding to a possible value of the truncated samples;
means for initializing the digital memory;
means for incrementing a value stored in the memory locations whose address correspond to a possible truncated sample value upon reception of a corresponding sample;
means for determining or estimating a maximum value stored within the digital memory;
means for determining the address of a memory location storing a value which is nearest to a predetermined fraction of the maximum value; and
means for computing a level of a noise affecting the input signal from the thus-determined address.

39. An electronic circuit comprising:
at least an input port for an analog input signal comprising signal pulses and noise;
means for sampling the analog input signal and converting it to digital format;
a digital band-pass filter matched to an expected shape of pulses contained within the signal, connected for filtering the digitized signal;
a device according to claim 38, receiving as its input port the filtered digitized signal;
means for computing a threshold level as a function of an estimated noise level outputted by the device;
comparator means of detecting a pulse whenever the digitized input signal crosses the threshold level; and
means extracting a set of samples of the digitized and filtered signal, having a predetermined size, corresponding to each detected pulse.

40. An implantable neurobiological recording system comprising:
a multi-electrode array for acquiring neural action potential signals;
an electronic circuit according to claim 39, receiving at its at least one input port signals acquired by the multi-electrode array; and
means for transmitting signals outputted by the electronic circuit to non-implantable external devices.

41. A method for classifying pulse signals comprising:
quantifying a set of predetermined features of a pulse signal to be classified;
representing the pulse signal as a point in a feature space containing a dynamically updated set of clusters of points, a subset of the clusters being considered as significant;
including the point representing the pulse signal in a nearest cluster—either significant or not—according to a predetermined metric, or create a new non-significant cluster centered on the point, if its distance from any existing cluster exceeds a threshold; and
classify this same point as being associated to a nearest significant cluster according to the metric; and
updating the set of clusters taking into account the thus-classified signal.

42. A method according to claim 41, wherein the clusters comprises a number of points, representing pulse signals, exceeding a predetermined threshold are considered as significant.

43. A method according to claim 41, wherein the updating the set of clusters comprises modifying the position and/or the size of the cluster in which the point has been included.

44. A method according to claim 43, wherein the updating the set of clusters further comprises merging overlapping clusters, or clusters whose distance is lower than a threshold.

45. A method according to claim 41, wherein the updating the set of clusters further comprises deleting at least one non-significant cluster, according to a priority criterion, if the number of clusters exceeds a predetermined threshold.

46. A method according to claim 41, wherein the features of a pulse signal to be classified comprise at least one of:
a distance between a minimum and a maximum of the pulse;
an area of one or more lobes of the pulse;
a pulse energy;
a peak-to-peak amplitude; and
a number of times the signal exceeds a predetermined threshold.

47. A method according to claim 41, wherein the pulse signals to be classified are neural action potential signals, and wherein each significant cluster is associated to a neuron whose action potential signals are acquired.

48. A method of processing a sampled and digitized pulse signal comprising:
detecting pulses by a method according to claim 33; and
classifying the detected pulses.

49. An electronic circuit according to claim 39, further comprising:
data processing means for classifying the detected pulses; and
means for outputting classification results and events affecting significant clusters.

50. An implantable neurobiological recording system comprising:
a multi-electrode array for acquiring neural action potential signals;
an electronic circuit according to claim 49, receiving at least one at its input port signals acquired by the multi-electrode array; and
means for transmitting signals representative of classification results and events affecting significant clusters, outputted by the electronic circuit, to non-implantable external devices.
